(19) United States
(12) Patent Application Publication
(10) Pub. No.: US 2007/0098653 A1
(43) Pub. Date: May 3, 2007

(54) Non-aqueous liquid composition having ultraviolet-absorbing capability

(76) Inventors: Mitsuo Tamasawa, Matsudo (JP); Hideo Yamaguti, Yoshio (JP); Atsushi Hasegawa, Abiko (JP)

Correspondence Address: MANELLI DENISON & SELTER, 2000 M STREET NW SUITE 700, WASHINGTON, DC, 20036-3307, US

(21) Appl. No.: 11/590,752

(22) Filed: Nov. 1, 2006

(30) Foreign Application Priority Data
Nov. 2, 2005 (JP) 2005-319266

(51) Int. Cl. A61K 8/89

(52) U.S. Cl. 424/59; 424/70.12

(57) ABSTRACT

The present invention provides a non-aqueous liquid composition from which the coated film on the skin is transparent and slippery, does not cause stickiness and uncomfortable feeling such as creaky feeling and stretch feeling, but causes fresh feeling of use, and keeps the capability of protecting the skin from ultraviolet rays for a desired period of time even under the presence of sweat or sebum, and which is suitable for sunscreen cosmetics, a skin external preparation or a bandage preparation. The non-aqueous liquid composition comprises particles composed of at least a copolymer derived from a silicone macromer (A) and an ultraviolet-absorbing compound and silicone oil in which the particles are dispersed, wherein the silicone macromer (A) exists in the copolymer within 1 to 20 wt % of all components of the particles and orients toward the outer surface of the particle, and wherein, the ultraviolet-absorbing compound is incorporated in the polymer particle as an independent component or a part of the copolymer.

### FIELD OF THE INVENTION

The present invention relates to a non-aqueous liquid composition having an ultraviolet-absorbing capability. More specifically, the present invention relates to a non-aqueous liquid composition in which organic particles having an ultraviolet-absorbing capability are dispersed in silicone oil.

### BACKGROUND OF THE INVENTION

In recent years, since it was reported that the dose of ultraviolet radiation arriving at surface of earth has increased due to ozone layer depletion, a social interest in defense from the ultraviolet radiation has been raised. With this view, various cosmetics, external preparations for skin and bandages to protect skin from ultraviolet rays, which may be hereafter sometimes abbreviated simply as cosmetics, have been put to practical use.

Conventionally, as for cosmetics capable of protecting skin from ultraviolet rays, the cosmetics containing an organic ultraviolet absorber such as benzotriazole derivative, benzophenone derivative, a salicylic acid derivative, a para-aminobenzoic acid derivative, a cinnamic acid derivative and an urocanic acid derivative, and an inorganic ultraviolet absorber such as particulate zinc oxide and particulate titanium oxide have been widely used.

In the conventional cosmetics containing an inorganic ultraviolet absorber, the ultraviolet absorber in the sufficient amount to absorb ultraviolet rays (mainly UV-A wave) caused the problems of creaky feeling and white blurring. On this point, various improvements are being suggested even now, but the problems have not been completely solved. In this regard, for instance, refer to JP 2003-327521A, JP 2004-277289A and JP 2002-265342A.

In the conventional cosmetics containing an organic ultraviolet absorber, the organic ultraviolet absorber in the amount required for a sufficient ultraviolet-absorbing effect resulted in a large amount of oil solvent. As a result, the conventional cosmetics had a disadvantage of sticky feeling.

In addition, even where containing any type of ultraviolet absorbers, the conventional cosmetics have a problem that the ultraviolet absorber exudes due to sweat or sebum, for example, when using them before doing sports. Thus, the conventional cosmetics could not keep the ultraviolet-absorbing capability for a desired period of time.

In the meantime, in the technical field of coating agents which are applied to substrates composed of various plastics, metals or glass, JP 09-3395A, of which the content is herein incorporated by reference, discloses a polymer type ultraviolet-absorbing lacquer composition which contains as an effective component a copolymer obtained by polymerizing 2-2′-hydroxy-5′-(methacryloyloxyethyl) phenyl)-2H-benzotriazol, which is an ultraviolet-absorbing monomer, with cyclohexyl methacrylate and methacrylic acid. A coating formed by the composition does not exude an ultraviolet-absorbing component and can thus retain weathering resistance for a long period.

However, in the composition, the copolymer is dissolved in a solvent for paint such as a thinner, and is not suitable for use in cosmetics.

JP 07-316242A, of which the content is herein incorporated by reference, discloses as a cosmetic material an aqueous emulsion which comprise polymer particles having a core-shell structure and containing an ultraviolet absorber in the core, which is manufactured by dripping a monomer solution containing an ultraviolet absorber dissolved therein into a solution containing a water-soluble resin neutralized by amine or ammonia and dispersed in water.

The emulsion is friendly to global environment, because it is an aqueous emulsion. In addition, the emulsion is superior in storage stability, because the trapping of an ultraviolet absorber in particles can prevent an ultraviolet absorber from exuding into water.

However, the emulsion contains particles having an acrylic water-soluble resin at the surface thereof, namely as a shell component, in water. Therefore, when the emulsion was applied to skin, a coating formed by the particles having the acrylic resin at the surface thereof was insufficient in terms of characteristics required for cosmetics such as slipperiness .

JP 2002-308909 A discloses a composition containing an oil-soluble ultraviolet absorber absorbed in silicone-modified polymer particles. However, the composition cannot contain a large amount of ultraviolet absorber, because the amount of ultraviolet absorber depends on the absorbability to the polymer particle. Further, the composition had a large particle size of 1 to 3 μm, and was insufficient in characteristics required for ultraviolet-blocking cosmetics such as sunscreen cosmetics and foundations.

JP 04-501076A discloses a composition for bandage obtained by dissolving 3-methacryloyloxypropyl (trimethylsiloxy)silane and methyl methacrylate in ethyl acetate, polymerizing them to produce a siloxane-containing polymer and dissolving it in volatile polydimethylsiloxane. The composition is substantially unstimulative, can be converted into a film at room temperature, and can form a bandage that is sticky and compatible to a user and permeates water vapor.

JP 02-247110A discloses makeup cosmetics dispersing a cosmetic powder in a mixture containing as essential components an acrylic-silicone graft copolymer, a low-viscosity silicone oil and a volatile hydrocarbon oil, wherein the acrylic-silicone graft copolymer is obtained by dissolving radical-polymerizable monomers mainly containing a specific dimethylpolysiloxane compound and an acrylate in toluene and then radically polymerizing the components. The makeup cosmetics have not only superior water resistance and perspiration resistance but also excellent oil resistance and sebum resistance, and further have abrasion resistance.

For the purpose of acquiring characteristics required for bandage and makeup such as water resistance, oil resistance and abrasion resistance, these composition and cosmetics are manufactured by dissolving a copolymer having a structural unit derived from a silicone monomer in high ratio, in the solvents as mentioned above.

On the other hand, the increased silicone monomer unit caused insufficiency in feeling of use required in many cosmetics.

More specifically, these composition and makeup cosmetics are obtained by polymerizing desired monomers in ethyl acetate or toluene, precipitating the obtained copolymer in hexane, drying it, and subsequently dissolving the copolymer in volatile polydimethylsiloxane or low-viscosity silicone oil.

Therefore, in order to dissolve the obtained copolymer in the solvents, it is required to control the ratio of a structural unit derived from a silicone monomer in the copolymer to 30 wt % or higher. In addition, in the composition and makeup cosmetics, polymer chains are extended in various directions and exist in a state of being intertwined with each other. For these reasons, these compositions and cosmetics do not spread well when being applied to skin, a dry coating formed by them on skin does not follow well the movement of the skin due to the intertwined macromolecule chains, and may give users unpleasant feeling such as feeling of stretch on the skin. It further was slow that the compositions and cosmetics were dried and they tend to be sticky, because of containing a structural unit derived from the silicone monomer at a high ratio in the copolymer and being highly soluble to a silicone-oil solvent.

On the other hand, JP 08-269332A discloses a non-aqueous dispersion for a cosmetic material which comprises a non-aqueous solvent containing silicone oil, a dispersion-stabilizing agent composed of a (co)polymer derived from specific silicon-containing monomers, and dispersed particles composed of a (co)polymer substantially insoluble in the non-aqueous solvent. The dispersion aimed at providing a cosmetic material having superior cosmetic functionality and usability, in addition to forming a coating with superior water resistance, oil resistance and physical properties for coating.

However, the non-aqueous dispersion contains a (co)polymer derived from silicon-containing monomer(s) as a dispersion-stabilizing agent, and dispersed particles per se do not have stably-dispersing capability. In addition, JP 08-269332A does not disclose at all to incorporate an ultraviolet-absorbing compound into the dispersed particle while making the dispersed particle per se have stably-dispersing capability in the silicone.

JP 2002-255748A, of which the content is herein incorporated by reference, discloses a process for producing a liquid non-aqueous dispersion wherein polymer particles are dispersed in silicone oil, the process comprising the step of one-step polymerizing a silicone macromer mainly containing dimethylpolysiloxane having a radical polymerizability with a group of a vinyl monomer mainly containing (meth)acrylate in the silicone oil under the absence of a dispersion-stabilizing agent. The process can provide a liquid non-aqueous dispersion for cosmetics which has excellent coatability and followability as well as excellent water resistance, oil resistance, and adhesiveness.

However, the process also does not intend at all to incorporate an ultraviolet-absorbing compound in particles consisting of a copolymer while making a silicone macromer exist at the surface of the particles, in a non-aqueous dispersion medium. Further, JP 2002-255748A does not disclose or suggest a non-aqueous composition stably dispersing particles having a multilayer-structure including a shell layer in which a silicone macromer dominantly exists at the surface of the particles and a core layer containing an ultraviolet-absorbing compound, in a non-aqueous dispersion medium.

### SUMMARY OF THE INVENTION

An object of the present invention is to provide a non-aqueous liquid composition for cosmetics, an external preparation for skin and a bandage which can keep the capability of protecting skin from ultraviolet rays for a desired period of time when being applied to skin, even under the presence of sweat or sebum, have transparent appearance, does not cause sticky feeling and an uncomfortable feeling such as creaky feeling and stretch feeling, and can have superior slipperiness.

In order to achieve the above described object, the present inventors tried radically polymerizing a vinyl monomer mainly containing a (meth)acrylate monomer and an organic ultraviolet absorbing agent or an ultraviolet-absorbing monomer having different polarities from that of silicone oil together with a small amount of the silicone macromer in silicone oil, through process of one or more steps. Then, the present inventors have found that the reaction results in a polymer particle in which organic ultraviolet absorbing agents are incorporated, or ultraviolet-absorbing monomers form a part of the copolymer, and silicone macromers exist in a state of orienting toward the outer surface of the polymer particle.

The present inventors have further found that the polymer particles having ultraviolet-absorbing capability are stably dispersed in silicone oil based on the structural features.

Thus, the present invention provides a non-aqueous liquid composition comprising particles at least composed of a copolymer containing a silicone macromer and an ultraviolet-absorbing compound, and silicone oil in which the particles are dispersed; wherein the silicone macromer is within 1 to 20 wt % of all components of the particle and orient toward the outer surface of the particle; and wherein the ultraviolet-absorbing compound is incorporated in the polymer particles as an independent component or a part of the copolymer.

In one embodiment, the present invention provides a non-aqueous liquid composition comprising particles having ultraviolet-absorbing capability and a silicone oil in which the particles are dispersed; wherein the particles contain a copolymer derived form a silicone macromer (A) and a vinyl monomer (B) mainly containing a (meth)acrylate monomer, and an organic ultraviolet absorber (C-1); and wherein the silicone macromer (A) is within 1 to 20 wt % of whole components of the particle and orient toward the outer surface of the particle.

In another embodiment, the present invention provides a non-aqueous liquid composition comprising particles having ultraviolet-absorbing capability and silicone oil in which the particles are dispersed; wherein the particles have a shell layer composed of a copolymer derived from a silicone macromer (A) and a vinyl monomer (B) mainly containing a (meth)acrylate monomer, and a core layer containing a (co)polymer mainly derived from a (meth)acrylate monomer and an ultraviolet absorber (C-1); and wherein the silicone macromer (A) is within 1 to 20 wt % of whole components of the particle and orient toward the outer surface of the particle.

In still another embodiment, the present invention provides a non-aqueous liquid composition comprising particles having ultraviolet-absorbing capability and a silicone oil in which the particles are dispersed; wherein the particles is composed of a copolymer derived from a silicone macromer (A), a vinyl monomer (B) mainly containing a (meth)acrylate monomer and an ultraviolet-absorbing monomer (C-2) ; and wherein the silicone macromer (A) is within 1 to 20 wt % of whole components of the particle and orient toward the outer surface of the particle.

In the other embodiment, the present invention provides a non-aqueous liquid composition comprising particles having ultraviolet-absorbing capability and a silicone oil in which the particles are dispersed; wherein the particles have a shell layer composed of a copolymer derived from a silicone macromer (A) and a vinyl monomer (B) mainly containing a (meth)acrylate monomer, and a core layer composed of a copolymer derived from a vinyl monomer (B) mainly containing a (meth)acrylate monomer and an ultraviolet-absorbing monomer (C-2); and wherein the silicone macromer (A) is within 1 to 20 wt % of whole components of the particle and orient toward the outer surface of the particle.

In the other embodiment, the present invention further provides a preferable method of producing a non-aqueous liquid composition containing particles having ultraviolet-absorbing capability and a silicone oil in which the particles are dispersed according to the present invention, which comprises subjecting 1 to 20 wt % of a silicone macromer (a), 30 to 98 wt % of a vinyl monomer (b) mainly containing (meth)acrylate, and 1 to 60 wt % of an organic ultraviolet absorber (c-1) or an ultraviolet-absorbing monomer (c-2), to a radical polymerization reaction of one or more steps in the silicone oil.

The expression of “(co)polymer” as used in the present specification refers to a polymer or a copolymer.

The term of “silicone macromer(s)” as used in the present specification refers to molecule(s) with a molecular weight of 1,000 or more having a dimethylpolysiloxane framework and a radical-polymerizable group as well.

The phrase of “vinyl monomer(s) mainly containing (meth)acrylate(s)” as used in the present specification refers to a group of vinyl monomer(s) mainly containing a monomer having a (meta)acryloyl group and optionally containing other vinyl monomers as well. A phrase of “mainly containing a (meth)acrylate monomer(s)” refers to containing a (meth)acrylate monomer(s) in 50 wt % or more, preferably 60 wt % or more, further preferably 70 wt % or more and particularly preferably 80 wt % or more, of the vinyl monomer(s). The term of “ultraviolet-absorbing monomer” as used in the present specification refers to monomer(s) having a functional group having ultraviolet-absorbing capability, in addition to a vinyl group. However, as used in the present specification, these monomers shall not include a monomer having a principal chain formed by a dimethylsiloxane bond.

These monomers preferably have a different polarity from that of a silicone macromer. Specifically, these monomers preferably have a surface tension of 28 dyne/cm² or larger and a solubility parameter of 8.5 or more. On the contrary, a silicone macromer as used in the present invention typically has a surface tension of 15 to 22 mN/m² and a solubility parameter of 7.1 to 7.5.

The term of “surface tension” as used in the present specification refers to a value measured with a ring method for a sample in liquid at room temperature by using, for instance, a measurement device manufactured by Kyowa Interface Science Co., LTD., or a value measured with a pendant drop method for a sample in solid at atmospheric temperature by using, for example, MODEL:CAM200 manufactured by Altech Alt Co., LTD. In addition, the term of “solubility parameter” as used in the present specification refers to a value calculated according to the expression of Fedors.

In addition, the term of “viscosity” as used in the present specification refers to a value measured with a BM type rotational viscometer (BM type viscometer MODEL BM made by Toki Sangyo Co., LTD.) under the conditions of a rotor No. 2 and 12 rpm for a sample solution left at 25° C. for two hours after the preparation.

Contrary to the above described definition, when a copolymer is referred to in the present specification, the term of “silicone macromer(s)” is regarded as a structural unit resulting from the silicone macromer(s) as defined above by a radical-polymerizable group therein being cleaved. Similarly, when a (co)polymer is referred to in the present specification, the term of “monomer(s)” is regarded as a structural unit resulting from monomer(s) as defined above by a radical-polymerizable group therein being cleaved.

The expression of “particles are stably dispersed in silicone oil” means that particles exist in silicone oil in a state of keeping a form of particle without agglomerating or being destroyed.

As described above, a liquid composition according to the present invention has the dispersed particles containing a copolymer derived from a silicone macromer in a small amount of 1 to 20 wt % in silicone oil. Then, the liquid composition can have very low viscosity while dispersing a large amount of the particles having ultraviolet-absorbing capability in a small amount of solvent. Accordingly, the liquid composition according to the present invention can reduce sticky feeling and have superior drying properties when being applied to skin. Further, the liquid composition according to the present invention comprises particles having such a structure that a silicone macromer orients toward the outer surface, as an effective component. Accordingly, the silicone macromer dominantly exists at the surface of the film as formed, and the film can give a user smooth feeling when being applied to skin.

In addition, in a liquid composition according to the present invention, an organic ultraviolet absorber is incorporated in a copolymer particle or an ultraviolet-absorbing monomer composes a part of a copolymer. Accordingly, the liquid composition can form a coating which does not exude an ultraviolet-absorbing component even when sweat or sebum exists around it, and keep desired ultraviolet-absorbing capability for a long period of time. In addition, in one embodiment of the present invention, particles having ultraviolet-absorbing capability can have a small diameter suitable for use in cosmetics: The smaller is the diameter, the smoother feeling is obtained when the cosmetics are applied to the skin.

Furthermore, in a liquid composition according to the present invention, a resin component having ultraviolet-absorbing capability in particulate form is dispersed in a solvent. Accordingly, the composition forms a coating in which individual particles having the ultraviolet-absorbing capability link with each other when being applied to skin. Therefore, the coating can have a shape precisely reflecting a fine shape such as the wrinkle of skin, and flexibly follow the movement of skin. Consequently, the liquid composition according to the present invention can form a coating film which gives users less uncomfortable feeling such as stretch feeling and also has superior adhesiveness to skin. The coating film also can reduce creaky feeling derived from an inorganic ultraviolet absorber.

A liquid composition according to the present invention also comprises a particle containing a copolymer derived from a vinyl monomer mainly containing a (meth)acrylate monomer. Accordingly, the composition can form a coating film which has superior transparency, and does not have a problem with white blurring caused by an inorganic ultraviolet absorber. Further, an ultraviolet absorbing compound retained in the film sufficiently can exert ultraviolet-absorbing properties due to the superior transparency.

### DESCRIPTION OF THE PREFERRED EMBODIMENTS

As described above, a liquid composition according to the present invention comprises dispersed particles which contain at least a copolymer derived from a silicone macromer (A) and a vinyl monomer (B) mainly containing (meth)acrylate, and an ultraviolet-absorbing compound (C), in silicone oil; wherein the silicone macromer (A) is within 1 to 20 wt % of whole components of the particles and orient toward the outer surface of the particles, and wherein the ultraviolet-absorbing compound (C) is incorporated in the polymer particle as an independent component or a part of the copolymer, namely derived from an ultraviolet-absorbing monomer. The liquid composition according to the present invention is more specifically described below.

I. Particle

I-1. Components of particle

In one embodiment, a particle in a liquid composition according to the present invention contains a copolymer derived from a small amount of silicone macromer (A) and a vinyl monomer (B) mainly containing (meth)acrylate, and an organic ultraviolet absorber (C-1).

In another embodiment, a particle in a liquid composition according to the present invention contains a copolymer derived from a small amount of a silicone macromer (A), a vinyl monomer (B) mainly containing a (meth)acrylate monomer and an ultraviolet-absorbing monomer (C-2).

In still another embodiment, a particle in a liquid composition according to the present invention has a shell layer containing a copolymer derived from a small amount of a silicone macromer (A) and a vinyl monomer (B) mainly containing a (meth)acrylate monomer, and a core layer containing a (co)polymer mainly derived from a (meth)acrylate monomer (B) and an ultraviolet absorber (C-1).

In yet another embodiment, a particle in a liquid composition according to the present invention has a shell layer containing a copolymer derived from a small amount of a silicone macromer (A) and a vinyl monomer (B) mainly containing a (meth)acrylate monomer, and a core layer containing a copolymer derived from a vinyl monomer (B) mainly containing a (meth)acrylate monomer and an ultraviolet-absorbing monomer (C-2).

In any embodiments of the present invention, the silicone macromer (A) plays an important role in separating an ultraviolet-absorbing compound from silicone oil while stably dispersing a particle in silicone oil. For the purpose, the silicone macromer (A) preferably has approximate polarity to and high affinity for silicone oil. Specifically, the silicone macromer preferably has a surface tension of 22 mN/m or lower (value measured before polymerization), and a solubility parameter of 7.1 to 7.5.

A silicone macromer (A) having such characteristics includes, as a typical example, a compound shown in the general formula:
[Formula 1]

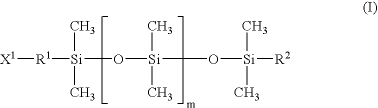

wherein, R¹ represents a linear or branched alkylene group having 1 to 10 carbon atoms, and preferably represents the linear or branched alkylene group having 1 to 4 carbon atoms;

R² represents a linear or branched alkyl group having 1 to 10 carbon atoms, and preferably represents the linear or branched alkyl group having 1 to 4 carbon atoms;

m represents an integer of 3 to 300, and preferably represents an integer of 50 to 200, because, when m in the above described formula (I) is less than 3, the particle lacks in dispersion stability, and when m exceeds 300, the obtained liquid composition tends to acquire high viscosity; and

x¹ represents a radical-polymerizable group including, for instance, an acryloxy group, a methacryloxy group, a styryl group, an allyl group, a vinylbenzyl group, a vinylether group, an acrylic amide group, a vinylalkylsilyl group and a vinylketone group.

A specific example of a silicone macromer shown in the above described formula (I) includes, for instance, dimethylpolysiloxypropylacrylic ester, dimethylpolysiloxypropylvinylbenzyl ether, dimethylpolysiloxypropylacrylamide and dimethylpolysiloxypropionic vinylbenzilate. Particularly, dimethylpolysiloxypropylacrylic ester or dimethylpolysiloxypropylmethacrylic ester is preferable in term of having adequate copolymerizability with a (meth)acrylate monomer.

In a preferred embodiment, a silicone macromer according to the present invention preferably has a number average molecular weight of 1,000 to 100,000, and more preferably 2,000 to 50,000, in reduced value of polystyrene measured by gel permeation chromatography before polymerization. When the number average molecular weight is 1,000 or less, the particle in a liquid composition may lack in dispersion stability, because the silicone macromer has insufficient affinity for silicone oil. When the number average molecular weight exceeds 100,000, it becomes difficult for a copolymer to form a desired particulate structure in the production process. Consequently, the silicone chains of the copolymer may be intertwined with each other, and the copolymer may become a dissolved state resulting in the high viscosity of the liquid composition.

In the present invention, a particle can contain a copolymer derived from one or more of the various silicone macromers as described above.

In a preferred embodiment of the present invention, a silicone macromer exists in a particle within 1 to 20 wt % of all the components of the particle, more preferably 2 to 15 wt %, and further preferably 3 to 12 wt %.

When the particle contains more than 20 wt % of silicone macromer, the copolymer derived form the silicone macromer may become dissolved in silicone oil, and accordingly it may be difficult to keep individual particulate form in silicone oil. As a result, when the composition is applied to skin, it may not spread well and cause stretch feeling. In addition, when the particle contains more than 20 wt % of silicone macromer (A), the particle has increased solubility to silicone oil. Consequently, the volatile of the silicone oil may be reduced, and it may cause the deterioration of drying properties and sticky feeling.

On the other hand, when the particle contains less than 1 wt % of silicone macromer (A), its affinity for the silicone oil is reduced, and it may make its dispersion stability in a solvent insufficient.

As seen from the forgoing, in the embodiment in which the particle is composed of a copolymer derived from a silicone macromer (A), a vinyl monomer (B) mainly containing a (meth)acrylate monomer and an ultraviolet-absorbing monomer (C-2), a silicone macromer (A) exists in the particle in a range of 1 to 20 wt % of the copolymer, and preferably 2 to 15 wt %.

In the embodiment in which the particle is composed of a copolymer derived from a silicone macromer (A) and a vinyl monomer (B) mainly containing a (meth)acrylate monomer, and an organic ultraviolet absorber (C-1), the silicone macromer (A) typically exists in a range of 1 to 40 wt % of the copolymer, preferably a range of 2 to 30 wt %, and further preferably a range of 4 to 25 wt %.

In the embodiment in which the particle has a core-shell structure, a silicone macromer (A) can exist in a shell layer in a wide range of amount. Specifically, the silicone macromer (A) typically exists in a range of 1 to 90 wt % of all the components of the shell layer, preferably a range of 2 to 70 wt %, and further preferably a range of 4 to 50 wt %.

In the present invention, a vinyl monomer (B) mainly containing (meth)acrylate is preferably required to be efficiently polymerized with a silicone macromer (A) while orienting the silicone macromer (A) toward the outer surface of the particle. Further, the vinyl monomer (B) is preferably required to have properties for efficiently incorporating an ultraviolet-absorbing monomer or an organic ultraviolet-absorbing material into the particle. Accordingly, in the present invention, the vinyl monomer (B) mainly containing (meth)acrylate preferably has a different polarity away from those of silicone oil (D) and the silicone macromer (A), and has low affinity for them. On the other hand, the vinyl monomer (B) preferably has high affinity for the ultraviolet-absorbing monomer or the organic ultraviolet-absorbing material.

Specifically, the vinyl monomer preferably has a surface tension of 28 mN/m or higher and a solubility parameter of 8.5 or more, and more preferably has the surface tension of 30 mN/m or higher and the solubility parameter of 9.0 or more, which are measured before polymerization.

A (meth)acrylate monomer having such properties includes, for instance, an alkyl (meth)acrylate such as methyl(meth)acrylate, ethyl(meth)acrylate, N-butyl(meth)acrylate, isobutyl(meth)acrylate, t-butyl(meth)acrylate, 2-ethylhexyl (meth)acrylate, stearyl(meth)acrylate, benzyl methacrylate and cyclohexyl(meth)acrylate; a (meth)acrylate containing a hydroxyl group, for example, a hydroxyalkyl(meth)acrylate such as 2-hydroxyethyl (meth)acrylate and 2-hydroxypropyl (meth)acrylate; a (meth)acrylate containing a carboxyl group such as acrylic acid, methacrylic acid and itaconic acid; a basic(meth)acrylate such as N-N-dimethylaminoethyl (meth)acrylate and N-N-diethyl (meth)acrylate; an epoxy-containing (meth)acrylate such as glycidyl(meth)acrylate; and a (meth)acrylate containing a hindered amine group such as Adekastab LA-87 and LA-82 (manufactured by Asahi Denka Kogyo K.K.).

In addition to the above compounds, the vinyl monomer having such properties can include, for instance, an aromatic vinyl monomer such as styrene, alphamethyl styrene and vinyltoluene; an unsaturated nitrile such as (meth)acrylonitrile; a vinyl ester such as vinyl acetate; and N-vinylpyrrolidone. The vinyl monomer can also include, for instance, a polyfunctional vinyl monomer such as divinylbenzene, trimethylolpropane tri(meth)acrylate and ethyleneglycol di(meth)acrylate.

According to the present invention, a copolymer can include one or more of the above described a (meth)acrylate monomer and other vinyl monomers. The copolymer may include other monomers so far as they do not impair features of the invention.

In any embodiments according to the present invention, a vinyl monomer (B) mainly containing a (meth)acrylate monomer preferably exists in a particle within a range of 20 to 98 wt % of all particle components, further preferably within a range of 25 to 80 wt %, and particularly preferably within a range of 30 to 70 wt %.

Accordingly, in the embodiment in which a particle is formed of a copolymer derived from a silicone macromer (A), a vinyl monomer (B) mainly containing a (meth)acrylate monomer and an ultraviolet-absorbing monomer (C-2), the vinyl monomer (B) preferably exists in a range of 20 to 98 wt % of the copolymer, further preferably a range of 25 to 80 wt %, and particularly preferably a range of 30 to 70 wt %.

In the embodiment in which a particle is composed of a copolymer derived from a silicone macromer (A) and a vinyl monomer (B) mainly containing a (meth)acrylate monomer, and an organic ultraviolet absorber (C-1), the vinyl monomer (B) typically exists in the copolymer in a range of 60 to 99 wt % of the copolymer, preferably a range of 70 to 98 wt %, and particularly preferably a range of 75 to 96 wt %.

In the embodiment in which a particle has a core-shell structure, in any of a shell layer and a core layer, a vinyl monomer (B) typically exits in each layer within 50 to 99 wt % of all the components of each layer, preferably 60 to 98 wt %, and further preferably 70 to 96 wt %.

When the amount of a vinyl monomer (B) mainly containing a (meth)acrylate monomer is less than each of the lower limits as described above, the particle may not acquire sufficient transparency and may reduce the ultraviolet-absorbing capability.

In the embodiment in which a particle contains an organic ultraviolet absorber (C-1), the organic ultraviolet absorber (C-1) is preferably required to be efficiently incorporated in the polymer particle without dissolving in silicone oil, during the particle formulation through a polymerization reaction.

Accordingly, the organic ultraviolet absorber (C-1) preferably has a different polarity from that of silicone oil and a silicone macromer (A), and has low affinity for them. On the other hand, the organic ultraviolet absorber (C-1) preferably has high affinity for a vinyl monomer (B) mainly containing a (meth)acrylate monomer.

Specifically, the organic ultraviolet absorber preferably has a surface tension of 28 mN/m or higher and a solubility parameter of 8.5 or more, and more preferably has a surface tension of 30 mN/m or higher and a solubility parameter of 9.0 or more.

The organic ultraviolet absorber having the above properties may include a commercially available product. Such commercially available organic ultraviolet absorbers may include, for instance, a benzophenone type, a dibenzoyl methane type, a benzotriazole type, a salicylic acid type, a para-aminobenzoic acid type, a cinnamon acid type and an urocanic acid type organic ultraviolet absorber.

More specifically, a benzophenone ultraviolet absorber having such properties may include, for instance, 2,4-dihydroxybenzophenone,.2,2-dihydroxy-4,4-dihydroxybenzophenone, 2.-hydroxy-4-methoxybenzophenone, 2-hydroxy-4-methoxy-4-methylbenzophenone, 2-hydroxy-4-methoxybenzophenone, 4-phenylbenzophenone, 2-ethylhexyl-4-phenylbenzophenon-2-carboxylate, 2-hydroxy-4-n-octoxybenzophenone and 4-hydroxy-3-carboxybenzophenone.

A dibenzoylmethane absorbing agent having such properties may include, for instance, butylmethoxydibenzoylmethane and 4-methoxy-4-t-butyldibenzoylmethane.

A benzotriazole ultraviolet absorber having such properties may include, for instance, 2,2-hydroxy-5-methylphenyl benzotriazole and 2-(2-hydroxy-5-t-octylphenyl) benzotriazole.

A salicylic acid ultraviolet absorber having such properties may include, for instance, menthyl salicylate, amyl salicylate, octyl salicylate, phenyl salicylate, benzil salicylate, p-isopropanolphenyl salicylate and p-isopropanolphenyl salicylate.

A para-aminobenzoic acid ultraviolet absorber having such properties may include, for instance, para-aminobenzoic acid, octyldimethylbenzoic acid, glyceryl para-aminobenzoic acid, ethyldihydroxypropyl para-aminobenzoic acid, N-ethoxylateethyl para-aminobenzoate, dimethylmethyl para-aminobenzoate and dimethyl-2-ethylhexyl para-aminobenzoate.

A cinnamic-acid ultraviolet absorber having such properties may include, for instance, octylmethoxy cinnamate, ethyl-4-isopropyl cinnamate, 2,4-ethyl-diisopropyl cinnamate, 2,4-methyl-diisopropyl cinnamate, n-propyl-p-methoxy cinnamate, isopropyl-p-methoxy cinnamate, isoamyl-p-methoxy cinnamate, 2-ethylhexyl-p-methoxy cinnamate, 2-ethoxyethyl-p-methoxy cinnamate and cyclohexyl-p-methoxy cinnamate.

An urocanic acid ultraviolet absorber having such properties may include, for instance, urocanic acid and ethylurocanic acid.

According to the present invention, a particle may contain one or more of the above described ultraviolet absorber, depending on the type and intended use of the cosmetics.

In a preferable embodiment in which a particle contains an ultraviolet absorber (C-1), the ultraviolet absorber (C-1) preferably exists in the particle within 1 to 60 wt % of all the components of the particle, further preferably 20 to 50 wt %, and particularly preferably 30 to 50 wt %.

When the particle contains less than 1 wt % of ultraviolet absorber (C-1), the particle may not exert a sufficient ultraviolet absorption characteristics. On the other hand, when the particle contains more than 60 wt % of ultraviolet absorber (C-1), the particle may not be stably dispersed, because the copolymer has reduced affinity for silicone oil.

Accordingly, in the embodiment in which a particle is composed of a copolymer derived from a silicone macromer (A) and a vinyl monomer (B) mainly containing a (meth)acrylate monomer, and an ultraviolet absorber (C-1), the ultraviolet absorber (C-1) typically exists in the particle to occupy 1 to 60 wt % of all the components of the particle, preferably 20 to 50 wt %, and further preferably 30 to 50 wt %.

In the embodiment in which a particle has a core-shell structure, an ultraviolet absorber (C-1) can exist in a core layer in a wide range of amount. Specifically, the ultraviolet absorber (C-1) can be within 1 to 70 wt % of all the components of the core layer, preferably 20 to 60 wt %, and further preferably 30 to 60 wt %.

As described above, a liquid composition according to the present invention has extremely low viscosity. Accordingly, the composition can contain a large amount of dispersed particles having ultraviolet-absorbing capability in silicone oil, while keeping excellent feeling of use.

In the embodiment in which an ultraviolet-absorbing monomer (C-2) composes a part of a copolymer, the monomer (C-2) is preferably required to be efficiently polymerized with a vinyl monomer (B) mainly containing a (meth)acrylate monomer without dissolving in silicone oil, during the particle formulation through a polymerization reaction.

Accordingly, the ultraviolet-absorbing monomer (C-2) preferably has a different polarity from those of the silicone oil and a silicone macromer (A), and has low affinity for them. On the other hand, the monomer (C-2) preferably has high affinity for a vinyl monomer (B) mainly containing a (meth)acrylate monomer.

Specifically, the ultraviolet-absorbing monomer preferably has a surface tension of 28 mN/m or higher and a solubility parameter of 8.5 or more, and more preferably has a surface tension of 30 mN/m or higher and a solubility parameter of 9.0 or more.

An ultraviolet-absorbing monomer having such properties includes, for instance, an ultraviolet-absorbing monomer wherein a radical-polymerizable group such as an acryloxy group, a methacryloxy group, a styryl group, an allyl group, a vinylbenzyl group, a vinylether group, an acrylic amide group, a vinylalkylsilyl group and a vinylketone group is added to the above described organic ultraviolet absorber such as a benzotriazole type, a dibenzoyl methane type, a benzophenone type, a salicylic acid type, a para-aminobenzoic acid type, a cinnamic acid type and an urocanic acid type absorber.

Preferably, in term of a wide ultraviolet-absorption wavelength region and adequate copolymerizability with (meth)acrylate, an ultraviolet-absorbing monomer may be a (meth)acrylate having a benzotriazole ultraviolet-absorbing group shown in the general formula (II):
[Formula 2]

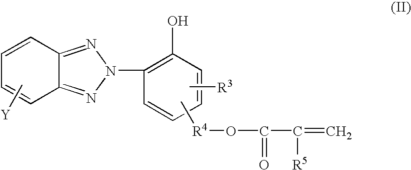

Wherein, R³ represents hydrogen atom or a hydrocarbon group having 1 to 8 carbon atoms, and preferably represents a hydrocarbon group having 1 to 4 carbon atoms;

R⁴ represents a linear or branched alkylene group having 1 to 6 carbon atoms, and preferably represents a linear or branched alkylene group having 1 to 3 carbon atoms;

R⁵ represents hydrogen atom or a methyl group; and

Y represents hydrogen atom or a halogen, an alkoxy group having 1 to 4 carbon atoms and preferably having 1 or 2 carbon atoms, a cyano group or a nitrile group.

A (meth)acrylate having a benzotriazole ultraviolet-absorbing group shown in the above general formula (2) includes, for instance, 2-(2′-hydroxy-5′-(methacryloyloxymethyl) phenyl)-2H-benzotriazole, 2-(2′-hydroxy-5′-(methacryloyloxyethyl)phenyl)-2H-benzotriazole, 2-(2′-hydroxy-5′-(methacryloyloxypropyl) phenyl)-2H-benzotriazole, 2-(2′-hydroxy-3-t-butyl-5′-(methacryloyloxyethyl)phenyl)-2-benzotriazole, 2-(2′-hydroxy-5′-t-butyl-3′-(methacryloyloxyethyl) phenyl)-2H-benzotriazole, 2-(2′-hydroxy-5′-(methacryloyloxyethyl)phenyl)-5-methoxy-2H-benzotriazole, and 2-(2′-hydroxy-5′-(methacryloyloxyethyl) phenyl)-5-t-butyl-2H-benzotriazole.

According to the present invention, a copolymer may be derived from one or more of the above described ultraviolet-absorbing monomers, depending on the type and the intended use of the composition.

In any embodiments in which a part of a copolymer is derived from an ultraviolet-absorbing monomer (C-2), the ultraviolet-absorbing monomer (C-2) preferably exists in the copolymer within 1 to 60 wt % of all the components of a particle, further preferably 20 to 50 wt %, and particularly preferably 30 to 50 wt %.

When the copolymer contains less than 1 wt % of ultraviolet-absorbing monomer (C-2), the copolymer may not exert a sufficient ultraviolet absorption characteristics. On the other hand, when the copolymer contains more than 60 wt % of ultraviolet-absorbing monomer (C-2), the particle may not be stably dispersed because the copolymer has reduced affinity for silicone oil.

Accordingly, in an embodiment in which a particle is formed of a copolymer derived from a silicone macromer (A), a vinyl monomer (B) mainly containing a (meth)acrylate monomer and an ultraviolet-absorbing monomer (C-2), the ultraviolet-absorbing monomer (C-2) typically exists in the copolymer within 1 to 60 wt % of the copolymer, preferably 20 to 50 wt %, and further preferably 30 to 50 wt %.

In an embodiment in which a particle has a core-shell structure, an ultraviolet-absorbing monomer (C-2) can exists in a core layer within 1 to 70 wt % of all the components of the core layer, preferably 20 to 60 wt %, and further preferably 30 to 60 wt %.

As described above, a liquid composition according to the present invention has extremely low viscosity. Accordingly, the composition can contain a large amount of dispersed particles having ultraviolet-absorbing capability in silicone oil, while keeping an excellent feeling of use.

In an embodiment in which a particle has a core-shell structure, the component ratio of a shell layer is preferably higher in term of keeping the dispersion stability of a particle. On the other hand, the component ratio of a core layer is preferably higher in term of increasing the amount of an ultraviolet-absorbing compound (C). In order to satisfy both of these requirements, the ratio of the shell layer to the core layer is preferably between 10 to 90 and 50 to 50 (W/W), further preferably between 15 to 85 and 40 to 60 (W/W), and particularly preferably between 20 to 80 and 35 to 65 (W/W).

In a preferable embodiment of the present invention, a (co)polymer composing a particle has a number average molecular weight preferably of 5,000 to 500,000, and particularly preferably of 10,000 to 100,000, in terms of a polystyrene reduced value measured by gel permeation chromatography, which are measured before polymerization.

When the molecular weight is 5,000 or less, a liquid composition may become sticky because of the decreased Tg of the (co)polymer. On the other hand, when the molecular weight is 500,000 or more, the particles results in reduced mutual fusion bonding by heat. Accordingly, a liquid composition may be insufficient in film-forming tendency, which is usually promoted by body temperature as applied to skin, and the adhesiveness of the coating film to skin.

In an embodiment of the present invention, a (co)polymer composing a particle preferably has a glass transition temperature of −30 to 150° C., and further preferably 10 to 100° C. When the (co)polymer has a glass transition temperature of −30° C. or lower, the liquid composition may become sticky. On the other hand, when the (co)polymer has a glass transition temperature of 150° C. or higher, the liquid composition may be insufficient in film-forming tendency, which is usually promoted by body temperature, and the adhesiveness of the film to the skin.

I-2. Structure of particle

According to the present invention, a particle contains a silicone macromer (A) in a small amount of 1 to 20 wt %, and the silicone macromers (A) orient toward the outer surface of the particle.

Accordingly, a particle containing a coating film component and an ultraviolet-absorbing component is dispersed in a solvent of silicone oil, keeping the independent particular form without dissolving in the solvent; while, the particle is stably dispersed in silicone oil, though containing just a small amount of a silicone macromer. Furthermore, the liquid composition can form a coating film superior in water repellency, oil repellency and slipperiness, because the particle has the silicone component at its surface.

According to the present invention, an organic ultraviolet absorber (C-1) or an ultraviolet-absorbing monomer (C-2) is interiorly incorporated into the particle as a part of the copolymer composing the particle or as an individual component of the particle.

Accordingly, the ultraviolet-absorbing component is almost separated from water or oil content outside the particle, and a coating film formed by the composition according to the present invention can keep ultraviolet absorptivity for a long time.

In an embodiment in which the liquid composition contains a particle having a core-shell structure, almost all silicone macromers (A) are concentrated in a shell layer of the particle, and exist therein so as to orient toward the particle surface. On the other hand, in the embodiment, organic ultraviolet absorbers are almost incorporated in a core layer of the particle, or ultraviolet-absorbing monomers almost constitute a part of polymer composing the core layer. Therefore, the core layer forms a multilayer structure consisting of two or more layers together with the shell layer as described above.

Thus, the particle of the embodiment further strengthens the excellent properties such as dispersion stability, water repellency, oil repellency, slipperiness and durable ultraviolet absorptivity as mentioned above.

According to the present invention, a silicone macromer predominantly exist at the surface of a particle. As a result, in a preferable embodiment, the particle has a contact angle with water of 90 to 120 degrees, and preferably of 90 to 110 degrees.

Such a contact angle with water is approximately the same as the contact angle of silicone oil with water used in the present invention, and is an index of the above described dispersion stability.

In the present invention, particles may have a desired particle size depending on the type and intended use of the composition. In an application to cosmetics, particles preferably have an average particle size of 0.05 to 2.0 μm, and more preferably of 0.05 to 0.5 μm. When the particles have the average diameter of 2 μm or larger, they may precipitate in a solution due to a specific gravity difference between the silicone oil and the particles, and may cause graininess and creaky feeling, particularly when abundantly applied onto skin.

II. Silicone oil

In the present invention, a silicone oil preferably has approximate polarity to that of the above described silicone macromer (A) and high affinity for the silicone macromer (A), so as to stably disperse particles having the silicone macromers (A) at the surface thereof. Specifically, the silicone oil preferably has a surface tension of 15 to 22 mN/m, a solubility parameter of 7.1 to 7.5, and a contact angle with water of 100 to 120 degrees.

A silicone oil having such properties includes, for instance, a dialkyl polysiloxane such as dimethylpolysiloxane, diethyl polysiloxane and dibutyl polysiloxane; an alkylphenyl polysiloxane such as methylphenyl polysiloxane and ethylphenyl polysiloxane; a cyclic dialkyl polysiloxane such as cyclic dimethyl polysiloxane, cyclic diethyl polysiloxane and cyclic dibutyl polysiloxane; a polysiloxane such as cyclic methylphenyl polysiloxane, cyclic ethylphenylpoly siloxane and cyclic butylphenyl polysiloxane; and a modified polysiloxane such as an amino-modified polysiloxane, a polyeter-modified polysiloxane and an alkyl-modified polysiloxane.

A dispersion medium may be one of those silicone oils singly, or two or more of them in combination, according to the use.

In an embodiment of the present invention, a dimethyl polysiloxane or a cyclic dimethyl polysiloxane is preferably used because of having approximate polarity and a similar composition to a silicone macromer. Particularly, a volatile dimethyl polysiloxane such as hexamethyl disiloxane or a volatile cyclic dimethyl siloxane such as decamethylcyclopenta siloxane is preferable because of having rapid drying property and low irritancy.

A liquid composition according to the present invention is characterized in that the particles with the special structure and composition as described above are dispersed in silicone oil. Accordingly, the composition can have far lower viscosity than that of a liquid composition wherein a resin is dissolved in a solvent. Specifically, the liquid composition, when having a solid content of 50 wt %, has the viscosity typically of 1,000 mPa·s or lower, preferably of 500 mPa·s or lower, and further preferably of 200 mPa·s or lower.

In a preferable embodiment of the present invention, the non-aqueous liquid composition can contain the silicone oil in an amount of 1 to 150 parts by weight to 100 parts by weight of the particles, and more preferably in an amount of 1 to parts by weight to 100 parts by weight of the particles.

III. Application

A liquid composition according to the present invention may be used singly or in combination with other components for cosmetics for sunscreen, a skin external preparation or a bandage preparation.

For instance, cosmetics for sunscreen can be prepared by adding an inorganic ultraviolet absorber, an organic ultraviolet absorber, a perfume, a surface active agent, a moisturizing agent, a thickener, a pH adjuster, lower alcohol, aliphatic hydrocarbon or an antiseptic agent to a liquid composition according to the present invention.

In this case, an inorganic ultraviolet absorber may include, for instance, a metallic oxide such as particulate zinc oxide, particulate titanium oxide and particulate cerium oxide. Similarly, an organic ultraviolet absorber may include, for instance, a cinnamic-acid ultraviolet absorber such as octylmethoxycinnamic acid; a salicyl acid ultraviolet absorber such as octyl salicylate; a benzoic acid ultraviolet absorber such as para-aminobenzoic acid; a triazine ultraviolet absorber such as bisresorcinylic triazine; and a benzophenone ultraviolet absorber such as 2,4-dihydroxy benzophenone. A perfume may include, for instance, a natural perfume such as lemon oil, orange oil and lime oil; and a synthetic perfume such as citronellyl acetate and amyl propionate. A surface active agent may include, for instance, a nonionic surface active agent such as polyoxyethylene alkyl ether; a cationic surface active agent such as lauryl amine oxide; and an anionic surface active agent such as sodium lauryl sulfate and alkyl sulfate triethanolamine ether. A moisturizing agent may include, for instance, polyethylene glycol, 1,3-butylene glycol, glycerine, erythritol, sorbitol and xylitol. A thickener may include, for instance, hydroxyethyl cellulose, carboxymethyl cellulose, tragacanth gum, chitin, chitosan and agar. A pH adjuster may include, for instance, a combination such as lactic acid—sodium lactate, citric acid—sodium citrate, and succinic acid—sodium succinate. A lower alcohol may include, for instance, ethanol, isopropanol, normal propanol, isobutyl alcohol and normal butyl alcohol. An aliphatic hydrocarbon may include, for instance, hexane, heptane, dodecane and cyclohexane. An antiseptic agent may include, for instance, alkyl para-hydroxybenzoate, sodium benzoate and potassium sorbate.

When the liquid composition is used for a skin external preparation for sunscreen, the preparation can be prepared by adding an antioxidant, an auxiliary antioxidant, a vitamin group, a skin-whitening agent, various extracts, a blood circulation accelerator and an antiinflammatory agent, to the cosmetics for sunscreen as described above.

In this case, an antioxidant may include, for instance, tocopherol, dibutyl hydroxytoluene and butylhydroxyanisol. Similarly, an auxiliary antioxidant may include, for instance, phosphoric acid, citric acid, ascorbic acid, malonic acid and succinic acid. A vitamin group may include, for instance, vitamin A, B1, B2, B6, C, E, and a derivative thereof; and pantothenic acid and a derivative thereof. A skin-whitening agent may include, for instance, a placenta extractant, a saxifrage extractant and arbutin. Various extractants include, for instance, an extractant of cork tree bark, Coptis Rhizome, Lithospermi Radix, a herbal preparation, sage, carrot, aloe, saffron, garlic, red pepper and seaweed. A blood circulation accelerator may include, for instance, nicotinic acid, capsaicin, tannic acid and acetylcholine. An antiinflammatory agent may include, for instance, tranexamic acid, thiotaurine and hypotaurine.

When a liquid composition according to the present invention is used for a bandage preparation for sunscreen, the preparation may be the liquid composition according to the present invention itself. Alternatively, in order to adjust a speed to be dried, the bandage preparation may be prepared by adding a solvent such as lower alcohol, hydrocarbon and silicone as described above to the liquid composition. In addition, the bandage preparation can be prepared by adding an antioxidant, an antioxidant auxiliary, a vitamin group, a skin-whitening agent, various extracts, a blood circulation accelerator or an antiinflammatory agent, as a skin external component, to the liquid composition.

IV. Production process

A liquid composition according to the present invention can be manufactured by subjecting 1 to 20 wt % of a silicone macromer (a), 20 to 60 wt % of a vinyl monomer (b) mainly containing a (meth)acrylate monomer, and 1 to 60 wt % of an organic ultraviolet absorber (c-1) or an ultraviolet-absorbing monomer (c-2), to a radical polymerization reaction of one or more steps in silicone oil.

In the production process, a silicone macromer (a) has approximate polarity to that of silicone oil (d), and has high affinity for the silicone oil. On the other hand, a vinyl monomer (b) mainly containing (meth)acrylate has a different polarity away from that of the silicone oil (d), and has low affinity for the silicone oil. An ultraviolet-absorbing monomer (c-2) and an organic ultraviolet absorber also have a different polarity away from that of the silicone oil (d), and have low affinity for the silicone oil. On the contrary, they have high affinity for the vinyl monomer (b).

Accordingly, when they are subjected to a radical polymerization reaction, the radical polymerization reaction proceeds in such a state that a silicone macromer (a) predominantly exists on a boundary surface to silicone oil and a vinyl monomer (b) mainly containing (meth)acrylate predominantly exists at a position separated from the silicone oil through the existence of the silicone macromer (a).

In addition, since an organic ultraviolet absorber and an ultraviolet-absorbing monomer have a different polarity from the silicone oil, an organic ultraviolet absorber is incorporated in a copolymer produced during the radical polymerization reaction, or the ultraviolet-absorbing monomer is polymerized at a position separated from the silicone oil through the existence of the a silicone macromer (a) and compose a part of the copolymer existing internally in the particle.

As a polymerization reaction proceeds, a produced copolymer gradually becomes insoluble in silicone oil depending on the composition ratio, is curled into a particulate form while orienting a silicone macromer (a) soluble in the silicone oil toward outside, and finally is dispersed in the silicone oil.

Consequently, according the process, a liquid composition in which particles containing an ultraviolet-absorbing compound are stably dispersed in silicone oil can be obtained without needing the addition of other components such as a dispersion-stabilizing agent.

In the meantime, even though having employed an aliphatic hydrocarbon with a low polarity, namely with a polarity harmonizing with a silicon macromer (a), such as hexane, which has surface tension of 17.9 mN/m and solubility parameter of 7.2, instead of silicone oil as a polymerization solvent, a stable particulate dispersion cannot be obtained as demonstrated in the example below.

Though a theoretical background of the event is unclear, it should be noted that a polymerization solvent used in a process according to the present invention is required to have not only approximate polarity but also a similar composition to those of the silicon macromer (a) which is a copolymerizing component. Thus, only silicone oil can be used as a polymerization solvent in the present invention until now.

The detail of each monomer, for example a type and a component ratio of monomer, corresponds to that as described in each component of a copolymerized particle according to the present invention.

In the present invention, there are no specific limitations to the presence or absence, or the type of a radical polymerization initiator. A general initiator may be used for it. Specifically, an initiator may include, but not limited to them, an azo compound such as 2,2, -azobisisobutyronitrile, 2,2-azobis (2,4-dimethylvaleronitrile), 2,2-azobis(-4 methoxy-2,4- dimethylvaleronitrile) and 2,2-azobisdimethyl isobutyrate; and an organized oxide such as lauroyl peroxide, t-butyl peroxide, dicumyl peroxide, t-butylperoxyl 2-hexanoate and benzoyl peroxide.

A polymerization initiator can be used singly or in combination of two or more initiators. The content is preferably 0.01 to 10 wt %, and further preferably is 0.1 to 3 wt % to the total amount of a monomer mixture.

In addition, it is acceptable for the purpose of adjusting a molecular weight to use a chain-transfer agent such as mercaptoacetic acid, mercaptopropionic acid, 2-propanethiol, ethylmercapto acetate, thiophenol, 2-naphthalenethiol and dodecyl melkapton.

In the polymerization reaction, a suitable reaction temperature is preferably selected depending on a monomer and solvent used, and a final product. Typically, it is preferable to perform the polymerization reaction at 30 to 180° C., and further preferable at 80 to 150° C. Under the temperature condition, the reaction can be completed in 5 to 10 hours in the presence of a polymerization initiator.

A polymerization method includes: (1) the batch polymerization method comprising the steps of previously mixing the whole quantity of the components (a), (b) and (c-1) or (c-2) in silicone oil, and adding and/or dripping a polymerization initiator to polymerize them;

(2) the dropping polymerization method comprising the step of dripping the whole quantity of the components (a), (b), (c-1) or (c-2), and a polymerization initiator into the silicone oil to polymerize them; and

(3) the batch/dropping polymerization method comprising the step of previously mixing a part of the components (a), (b) and, (c-1) or (c-2) in the silicone oil, and then, for example continuously, dripping and/or adding the rest of the components (a), (b) and (c-1) or (c-2), and the polymerization initiator to polymerize them.

In any of the above process, the dispersion in silicone oil of the particles wherein a silicone macromer orients toward the external surface thereof can be obtained by appropriately selecting a monomer composition and composition ratio.

A process of producing a particle having a core-shell structure may comprise the steps: firstly reacting the whole quantity of a component (a) with a part of component (b) in silicone oil to form a silicone-macromer-rich copolymer as a shell component easily dissolvable in the silicone oil; and then dripping by one or more step the rest of the component (b) and the ultraviolet absorber of the component (c-2) to form a core part. The process can results in a dispersion wherein copolymer particles containing an organic ultraviolet absorber are further stably dispersed.

According to the method, it is possible to form a particle having a multilayer structure consisting of three or more layers, by carrying out a process for forming the shell layer and/or the core layer in two or more times. At this time, by changing the composition ratio stepwise or continuously in the above process, it is also possible to form a particle in which a composition ratio gradually changes stepwise or continuously from the inside to the outside.

In a process according to the present invention, the dispersion stability and particle diameter of a particle may be mainly changed depending on a difference of polarity between a silicone macromer (a), and another monomer (b) and an organic ultraviolet absorber (c-1) or an ultraviolet-absorbing monomer (c-2), and a difference of affinity for silicone oil between those monomers. Accordingly, the particle diameter of the dispersed particle may be controlled by appropriately selecting the type of the silicone oil; the ratio among the silicone macromer (a), a vinyl monomer (b) mainly containing a (meth)acrylate monomer and the ultraviolet absorber (c-1) or the ultraviolet-absorbing monomer (c-2); and the type of these components.

The particle diameter of the dispersed particle can be adjusted also by controlling a polymerization reaction temperature, the type and amount of a polymerization initiator and other polymerization factors.

### EXAMPLE

The present invention will be specifically described with reference to the examples below. But, these examples are not intended to limit the present invention.

Production of liquid composition

### Example 1

Into a reaction vessel having a thermometer, a reflux condenser, a stirrer and a dropping funnel, 500 g of commercially available hexamethyldisiloxane (KF-96 with 0.5 Cs manufactured by Shin-Etsu Chemical Co., Ltd.) was charged as a solvent. The atmosphere was replaced by a nitrogen gas, and the solvent was heated to 95° C.

By mixing 250 g of methyl methacrylate and 100 g of 2-ethylhexyl acrylate as a (meth)acrylate monomer; 50 g of commercially available polydimethylsiloxypropyl acrylate (FM-0721 of mono-terminal type Silaplane with average molecular weight of 5,000 manufactured by Chisso Corporation) as a silicone macromer; 70 g of octylmethoxy cinnamate (PARSOL MCX manufactured by ROSCH Corporation) and 30 g of butylmethoxydibenzoyl methane (PARSOL 1789 manufactured by ROSCH Corporation) as an UV-absorbing agent; and 10 g of commercially available lauroyl peroxide (Peroyl L manufactured by Nippon Oil & Fats Co., Ltd.) as a polymerization initiator, a raw solution was prepared.

The raw solution was dripped to hexamethyldisiloxane for 180 minutes while keeping it at 95° C. Subsequently, the solution was heated to 100° C., and a solution prepared by dissolving 4 g of lauroyl peroxide in 50 g of hexamethyldisiloxane (KF-96 with 0.5 cs) was dripped to the solution for 120 minutes while keeping it at the same temperature. Further, the solution is kept at the same temperature for 120 minutes and then cooled to 50° C. to obtain a polymer dispersion with milky white which has a solid content of 50.1% and a viscosity of 180 mPa·s.

A particle of the obtained dispersion was composed of a polymer with an average molecular weight of 45,000, as measured by GPC method with SHODEX KF-80M manufactured by Showa Denko Corporation. In addition, the particle had an average particle diameter of 0.25 μm as measured with a laser doppler/frequency analysis type particle size distribution meter (UPA150 manufactured by Nikkiso Corp.). When the particle was observed with a scanning electron microscope (VE-8800 manufactured by Keyence Corporation Co., Ltd.), it was confirmed to be a spherical particle with a diameter of 0.2 to 0.4 μm.

In addition, in order to confirm a composition of a particle in the obtained dispersion, the dispersion is diluted with hexamethyldisiloxane two times and is centrifuged with a high-speed centrifugal separator (HIMAC CF-15R manufactured by Hitachi Koki Co., Ltd.) at 10,000 rpm to obtain a precipitate. Then, the precipitate was under a reduced pressure to obtain a white powder. The powder in an amount of 1 g was further dissolved in 1 g of ethanol and the solution was diluted with 10 g of methanol. Then, the solution was separated into a dissolving component and a precipitated component. The dissolving component in methanol was under a reduced pressure to eliminate methanol, and 0.18 g of a liquid substance was obtained. The liquid substance was applied onto a KBR plate, and was dried. An infrared absorption spectrum for the dried substance was measured with an IR7000 type infrared rays absorptiometer manufactured by JASCO Corporation. The spectrum conformed to the infrared absorption spectrum of a standard mixture containing PARSOL MCX and PARSOL 1789 at a weight ratio of 7:3. Consequently, it was confirmed that the particle contain an ultraviolet absorber.

### Example 2

500 g of decamethylcyclopenta siloxane (KF-995 manufactured by Shin-Etsu Chemical Co., Ltd.) is charged as a solvent into the reaction vessel, similarly with Example 1.

By mixing 80 g of methyl methacrylate and 20 g of 2-ethylhexyl acrylate as a (meth)acrylate monomer; 50 g of commercially available polydimethylsiloxypropyl acrylate (FM-0725 of mono-terminal type Silaplane with average molecular weight of 10,000 manufactured by Chisso Corporation) as a silicone macromer; and 2.5 g of commercially available tertiarybutoxyperoxyl-2-ethyl hexanoate (Perbutyl 0 manufactured by Nippon Oil & Fats Co., Ltd.), a raw solution for a shell was prepared.

The atmosphere in the reaction vessel was replaced with nitrogen gas, and the raw solution for the shell was heated to 105° C. and kept for 120 minutes at the same temperature to polymerize them as the first polymerization step.

Subsequently, By mixing 180 g of methyl methacrylate and 20 g of 2-ethylhexyl acrylate as a (meth)acrylate monomer; 100 g of octylmethoxy cinnamate (PARSOL MCX manufactured by ROSCH Corporation) and 50 g of butylmethoxydibenzoyl methane (PARSOL 1789 manufactured by ROSCH Corporation) as an UV-absorbing agent; and 3.0 g of tertiarybutoxyperoxyl-2-ethyl hexanoate (Perbutyl 0 manufactured by Nippon Oil & Fats Co., Ltd.) as a polymerization initiator, a mixture solution was prepared. The mixture was dripped into the reaction vessel for 120 minutes, while keeping the mixture at the same temperature.

Furthermore, when 60 minutes passed after the dropping, a solution of 4 g of tertiarybutoxyperoxyl-2-ethyl hexanoate in 50 g of decamethylcyclopenta siloxane was slowly dripped into the reaction vessel for 120 minutes to polymerize them as second polymerization step, while keeping the mixture at the same temperature.

Subsequently, the solution is heated to 120° C., further be left at the same temperature for 120 minutes and then is cooled to 50° C. to obtain a milky white polymer dispersion with a solid content of 50.2% and a viscosity of 90 mPa·s. A particle in the obtained dispersion was composed of a polymer with a weight average molecular weight of 55,000, had an average particle size of 0.25 μ and had a spherical shape.

In order to confirm a composition of a particle in the obtained dispersion, the dispersion is diluted with hexamethyldisiloxane two times and is centrifuged with a high-speed centrifugal separator at 10,000 rpm to prepare a precipitate. The precipitate was under a reduced pressure to obtain a white powder. The powder in an amount of 1 g was further dissolved in 1 g of ethanol and the solution was diluted with 10 g of methanol. Then, the solution was separated into a dissolving component and a precipitated component. The dissolving component in methanol was under a reduced pressure to eliminate methanol, and 0.28 g of a liquid substance was obtained. The liquid substance was applied onto a KBR plate, and was then dried. An infrared absorption spectrum for the dried substances was measured. The obtained spectrum coincided with the infrared absorption spectrum of a standard mixture containing PARSOL MCX and PARSOL 1789 at a weight ratio of 2:1. Consequently, it was confirmed that the particle contains an ultraviolet absorber.

### Example 3

According to the procedure in Example 1, 500 g of dimethylpolysiloxane (KF-96 1CS manufactured by Shin-Etsu Chemical Co., Ltd.) was charged as a solvent into a reaction vessel.

Then, by adding thereto 100 g of methyl methacrylate and 150 g of 2-ethylhexyl acrylate as a (meth)acrylate monomer; 50 g of polydimethylsiloxypropyl acrylate as a silicone macromer; and 200 g of commercially available 2-(2′-hydroxy-5′-(methacryloyloxyethyl)phenyl)-2H-benzotriazole (RUVA-93 manufactured by Otsuka Chemical Corp.) as an UV absorptive monomer having a radical polymerization group; and 7.5 g of tertiarybutoxyperoxyl-2-ethyl hexanoate, a raw solution was prepared.

The raw solution was put into the reaction vessel, and the atmosphere was replaced with nitrogen gas. Then, the raw solution was heated to 105° C., and was kept at the same temperature. When 180 minutes passed, a solution of 4 g of tertiarybutoxyperoxyl-2-ethyl hexanoate dissolved in 50 g of decamethylpenta siloxane was slowly dripped into the reaction vessel for 120 minutes, while keeping the mixture at the same temperature.

Then, the solution is heated to 120° C., further kept at the same temperature for 120 minutes and then cooled to 50° C. to obtain a milky white polymer dispersion with a solid content of 50.2% and a viscosity of 320 mPa·s.

A particle of the obtained dispersion was composed of a polymer with a weight average molecular weight of 86,000, had an average particle size of 0.35 μm and had a spherical shape.

### Example 4

500 g of decamethylcyclopenta siloxane as a solvent was charged into a reaction vessel, similarly with Example 1. By adding thereto 150 g of methyl methacrylate and 100 g of 2-ethylhexyl acrylate as a (meth)acrylate monomer; 100 g of commercially available polydimethylsiloxypropyl acrylate (FM-0725 of mono-terminal type Silaplane with average molecular weight of 10,000 manufactured by Chisso Corporation) as a silicone macromer; 150 g of 2-(2′-hydroxy-5′-(methacryloyloxyethyl) phenyl)-2H-benzotriazole as an UV absorptive monomer having a radical polymerization group; and 7.5 g of tertiarybutoxyperoxyl-2-ethyl hexanoate as a polymerization initiator, a raw solution was prepared.

The raw solution was put into a reaction vessel, and the atmosphere was replaced by nitrogen gas. Then, the raw solution was heated to 105° C., and was kept at the same temperature. When 180 minutes passed, a solution of 4 g of tertiarybutoxyperoxyl-2-ethyl hexanoate dissolved in 50 g of decamethylcyclopenta siloxane was slowly dripped into the reaction vessel for 120 minutes, while keeping the mixture at the same temperature. The solution was kept at the same temperature for 120 minutes and then cooled to 50° C. to obtain a milky white polymer dispersion with a solid content of 50.2% and a viscosity of 460 mPa·s.

A particle in the obtained dispersion was composed of a polymer with a weight average molecular weight of 72,000, had an average particle size of 0.21 μm and had a spherical shape.

### Comparative Example 1

A raw solution was prepared by polymerizing the same materials in the same condition as in Example 1 except that an aliphatic hydrocarbon mixture solvent (Isopar G manufactured by ExxonMobil Chemical Company) was used instead of hexamethyldisiloxane as a solvent.

The raw solution was dripped into Isopar G, while keeping the mixture at 95° C. However, in about 120 minutes after having started dripping, the reacted liquid began to become clouded, and subsequently formed precipitates and deposits. Thus, a polymer could not be obtained. Polymerization conditions are summarized in Table 1.

### Comparative Example 2

A raw solution was prepared by polymerizing the same materials in the same condition as in Example 4 except that heptane was used as a solvent instead of decamethylpentacyclosiloxane.

However, in about 100 minutes after having started addition, the reaction liquid began to become clouded, and subsequently formed precipitates and deposits. Thus, a polymer could not be obtained. Polymerization conditions are summarized in Table 1.

### Comparative Example 3

A raw solution was prepared by polymerizing the same materials in the same condition as used in Example 3 except that a charged amount of polydimethylsiloxypropyl acrylate (FM-0725) was changed to 4 g from 50 g and a charged amount of methyl methacrylate was changed to 146 g from 100 g.

In 90 minutes after having heated the raw solution to 105° C., the raw solution began to be deposited on the wall surface of a flask, and subsequently precipitated. Thus, a polymer could not be obtained.

### Comparative Example 4

As a solvent, 500 g of decamethylcyclopenta siloxane was put into a reaction vessel, similarly with Example 1. An atmosphere in the vessel was replaced with nitrogen gas; and then the solvent was heated to 95° C.

By mixing 180 g of methyl methacrylate and 20 g of 2-ethylhexyl acrylate as a (meta)acrylate monomer; 200 g of commercially available polydimethylsiloxypropyl acrylate (FM-0725 of mono-terminal type Silaplane with average molecular weight of 5,000 manufactured by Chisso Corporation) as a silicone macromer; 70 g of octylmethoxy cinnamate (PARSOL MCX manufactured by ROSCH Corporation) and 30 g of butylmethoxydibenzoyl methane (PARSOL 1789 manufactured by ROSCH Corporation) as an UV-absorbing agent; and 7.5 g of tertiarybutoxyperoxyl-2-ethyl hexanoate as a polymerization initiator, a raw solution was prepared. The raw solution was put into the reaction vessel, and the atmosphere was replaced with nitrogen gas. Then, the raw solution was heated to 105° C., and was kept at the same temperature. When 180 minutes passed, a solution of 4 g of tertiarybutoxyperoxyl-2-ethyl hexanoate dissolved in 50 g of decamethylcyclopenta siloxane was slowly dripped into the reaction vessel for 120 minutes, while keeping the mixture at the same temperature.

Subsequently, the solution is heated to 120° C., further kept at the same temperature for 120 minutes and then cooled to 50° C. to obtain a white polymer dispersion with a solid content of 50.3% and a viscosity of 660 mPa·s.

A particle in the obtained dispersion was composed of a polymer with a weight average molecular weight of 36,000, had an average particle size of 0.21 μm and had a spherical shape. The obtained liquid composition was dried for three days at room temperature, but kept a liquid state and did not form a coating film.

### Comparative Example 5

500 g of toluene, instead of decamethylcyclopenta siloxane used in Example 2, was put into a reaction vessel as a solvent, similarly with Example 2. The atmosphere was replaced with nitrogen gas, the raw solution was heated to 105° C. and kept at the same temperature.

Then, by mixing 100 g of methyl methacrylate and 100 g of 2-ethylhexyl acrylate as a (meth)acrylate monomer; 200 g of commercially available polydimethylsiloxypropyl acrylate (FM-0725 of mono-terminal type Silaplane with average molecular weight of 10,000 manufactured by Chisso Corporation) as a silicone macromer; 100 g of 2-(2′-hydroxy-5′-(methacryloyloxyethyl) phenyl)-2H-benzotriazole as an UV absorptive monomer having a radical polymerization group; and 7.5 g of tertiarybutoxyperoxyl-2-ethyl hexanoate as a polymerization initiator, a raw solution was prepared.

The raw solution was dripped to toluene for 180 minutes while keeping the mixture at 105° C. Further, a solution of 4 g of tertiarybutoxyperoxyl-2-ethyl hexanoate dissolved in 100 g of toluene was slowly dripped to the above mixture for 60 minutes while keeping the mixture at the same temperature.

Subsequently, the solution was heated to 110° C., further kept at the same temperature for 110 minutes and then cooled to 50° C. to obtain a white polymer dispersion with a solid content of 50% and a viscosity of 2,600 mPa·s.

An obtained translucent liquid in an amount of 100 g was poured into 1,000 g of methanol to thereby precipitate a polymer. The precipitate was separated, dried and then dissolved in decamethylcyclopenta siloxane to obtain a resin solution with a solid content of 50.2% and a viscosity of 8,600 mPa·s.

An obtained polymer had a weight average molecular weight of 43,000. The polymer was measured with a particle size distribution meter and observed with a scanning electron microscope, but the existence of a particle could not be confirmed.

### Comparative Example 6

500 g of toluene, instead of decamethylcyclopenta siloxane used in Example 2, was charged into a reaction vessel as a solvent, similarly with Example 1.

By adding thereto 75 g of methyl methacrylate and 75 g of 2-ethylhexyl acrylate as a (meth)acrylate monomer; 250 g of 3-methacryloyloxy (trimethylsiloxy) silane (TRIS) as a silicone macromer; 100 g of 2-(2′-hydroxy-5′-(methacryloyloxyethyl)phenyl)-2H-benzotriazole as an UV absorptive monomer having a radical polymerization group; and 7.5 g of tertiarybutoxyperoxyl-2-ethyl hexanoate as a polymerization initiator, a raw solution was prepared.

The atmosphere in the reaction vessel was replaced with nitrogen gas. Then, the raw solution was heated to 105° C., and kept at the same temperature. When 180 minutes passed, a solution of 4 g of tertiarybutoxyperoxyl-2-ethyl hexanoate dissolved in 50 g of toluene was slowly dripped into the raw solution for 120 minutes, while keeping the mixture at the same temperature. Further, the mixture was kept at the same temperature for 120 minutes, and then was cooled to 50° C. to thus obtain a translucent solution.

An obtained resin solution in the amount of 100 g was poured into methanol in the amount of 1,000 g to precipitate a polymer. The precipitate was separated, dried and then dissolved in hexamethyldisiloxane to obtain a translucent solution with a solid content of 50.3% and a viscosity of 4,300 mPa·s.

An obtained polymer had a weight average molecular weight of 56,000. The polymer was measured with a particle size distribution meter and observed with a scanning electron microscope, but the existence of a particle could not be confirmed.

Polymerization conditions and basic characteristics of the obtained liquid composition are respectively summarized in Table 1 and Table 2.

TABLE 1
Polymerization condition

| | | | | | | Comparative | | | | | |
|---|---|---|---|---|---|---|---|---|---|---|---|
| | | Example 1 | 2 | 3 | 4 | Example 1 | 2 | 3 | 4 | 5 | 6 |
| solvent | decamethyl- | | 500 | | 500 | | | | 500 | | |
| | cyclopenta siloxane | | | | | | | | | | |
| | hexamethyl- | 500 | | | | | | | | | |
| | disiloxane | | | | | | | | | | |
| | dimethyl- | | | 500 | | | | 500 | | | |
| | polysiloxane | | | | | | | | | | |
| | 1cs | | | | | | | | | | |
| | isopar g* | | | | | 500 | | | | | |
| | heptane | | | | | | 500 | | | | |
| | toluene | | | | | | | | | 500 | 500 |
| silicone | FM-0721 | 50 | | | | 50 | | | | | |
| macromer | FM-0725 | | 50 | 50 | 100 | | 100 | 4 | 200 | 200 | |
| | TRIS | | | | | | | | | | 250 |
| monomer | MMA | 250 | 260 | 100 | 150 | 250 | 150 | 146 | 180 | 100 | 75 |
| | 2EHA | 100 | 40 | 150 | 100 | 100 | 100 | 150 | 20 | 100 | 75 |
| ultraviolet | PARSOL MCX | 70 | 100 | | | 70 | | | 70 | | |
| absorber | PARSOL 1789 | 30 | 50 | | | 30 | | | 30 | | |
| | RUVA-93 | | | 200 | 150 | | 150 | 200 | | 100 | 100 |

| | | | | | | | | | | |
|---|---|---|---|---|---|---|---|---|---|---|
| polymerization initiator PBO | | 7.5 | 7.5 | 7.5 | | 7.5 | 7.5 | 7.5 | 7.5 | 7.5 |
| Peroyl L | 10 | | | | 10 | | | | | |
| reaction temperature(°) | 95 | 105 | 105 | 105 | 95 | 105 | 105 | 105 | 105 | 105 |
| polymerization method | dropwise | C/S | charging | charging | dropwise | charging | charging | charging | dropwise | charging |

TABLE 2
basic characteristic of liquid composition

| | | | | | Comparative | | |
|---|---|---|---|---|---|---|---|
| | Example 1 | 2 | 3 | 4 | Example 4 | 5 | 6 |
| solid content (%) | 50.1 | 50.2 | 50.2 | 50.2 | 50.3 | 50.2 | 50.3 |
| viscosity (mpa · s) | 180 | 90 | 320 | 460 | 660 | 8600 | 4300 |
| average particle size | 0.25 | 0.25 | 0.35 | 0.21 | 0.21 | — | — |
| weight average molecular weight | 45000 | 55000 | 86000 | 70000 | 36000 | 43000 | 56000 |
| silicone macromer wt % | 10% | 10 | 10 | 20 | 40 | 50 | 50 |
| ultraviolet absorber (monomer) | 20% | 30 | 40 | 30 | 20 | 20 | 20 |
| wt % | | | | | | | |

Performance Evaluation

The following tests were conducted on the liquid compositions obtained in Examples 1 to 4 and Comparative Examples 4 to 6, in order to evaluate the performance of them as an ultraviolet absorber.

(Testing Method)

1) Contact angle with water

Each obtained liquid composition was applied onto a transparent glass plate with an applicator so as to form a dried film with the thickness of 10 μm, and was dried at 35° C. One water droplet was dropped on the dried film and was photographed from a horizontal direction with Altech Alt Co., LTD, MODEL:CAM200. The contact angle was measured with Altech Alt Co., LTD, MODEL:CAM200.

2) Ultraviolet absorptivity

Each obtained liquid composition was applied onto a transparent glass plate with a bar coater to form films with several thicknesses. At the time when the transmittance of lights with the wavelengths of 290 to 380 nm, as measured with an automatic recording spectrophotometer (U-3400 manufactured by JASCO Corporation, became 0%, a thickness of the film was detected

3) Drying property

Each obtained liquid composition was applied onto a glass plate with a bar coater to try to form a dry film with a thickness of 2 μm. We then detected a necessary period of time to become raised no sticky feeling due to the volatilization of the solvent was measured.

4) Transparency

Each obtained liquid composition was applied onto a transparent glass plate with an applicator and was dried at 35° C., to form a dried film with the thickness of 20 μm. Then, the cloudiness of the film was judged by visual inspection.

The film showing transparency and no cloudiness was evaluated to be Excellent, ⊚; the film showing somewhat matte appearance and degraded transparency was evaluated to be Good, ◯; and the film showing opaque appearance due to cloudiness was evaluated to be Poor, ×.

5) Water resistance

Each obtained liquid composition was applied onto a cleaned fluororesin film with an applicator, and dried. Then, a formed film was peeled off, and was immersed in water for three days. The appearance change of a film was judged through visual inspection.

The film having shown no change was evaluated to be Excellent, ⊚; the film of which a part had dissolved was evaluated to be Good, ◯; and the film which had completely dissolved and had not had left at all was evaluated to be Poor, ×.

6) Oil resistance

A film obtained according to the above described item (5) was immersed in n-dodecane for 24 hours, and the change of the film state was judged through visual inspection.

The film having shown no change was evaluated to be Excellent, ⊚; the film of which a part had dissolved was evaluated to be Good, ◯; and the film which had completely dissolved and had not had left at all was evaluated to be Poor, ×.

7) Adhesiveness to skin

Several droplets of each obtained liquid composition were applied onto the back of a hand with a finger, and dried. Then, the formed film was rubbed with a finger. Then, a degree of adhesiveness of the film was judged from the peeled state of the film.

When the film was not peeled off even by rigorous rubbing, the adhesiveness was evaluated to be Excellent, ⊚; when a part of the film was peeled off by three times and more of rubbing, the adhesiveness was evaluated to be Good, ◯; and when the film was completely peeled off by one or two times of rubbing, the adhesiveness was evaluated to be Poor, ×.

8) Spreadability

Several droplets of each obtained liquid composition were applied onto the back of a hand with a finger, and were spread with a finger until the liquid was dried. The spreadability was evaluated by whether or not there was a caught feeling as spread.

When there was no caught feeling, the spreadability was evaluated to be Excellent, ⊚; when there was some caught feeling ( [symbol] ) but the composition could be spread, the spreadability was evaluated to be Good, ◯; and when there was caught feeling immediately after the composition started to be spread with a finger, and the composition could not be spread, the spreadability was evaluated to be Poor, ×.

9) Stretch feeling

Several droplets of each liquid composition were applied onto the back of a hand, and were naturally dried to form a coating film. Then, the stretch feeling was evaluated by a sense of a subject.

When there was no stretch feeling, the stretch feeling was evaluated to be Excellent, ⊚; when the subject felt uncomfortable due to some stretch feeling, the stretch feeling was evaluated to be Good, ◯; and when the subject felt strong stretch feeling, the stretch feeling was evaluated to be Poor, ×.

TABLE 3
evaluation result

| | | | | | Comparative | | |
|---|---|---|---|---|---|---|---|
| | Example 1 | 2 | 3 | 4 | Example 4 | 5 | 6 |
| contact angle with water | 98 degrees | 100 | 95 | 99 | 100 | 98 | 96 |
| ultraviolet absorptivity | 3.2μ | 2.1 | 1.4 | 2.1 | 3.2 | 3.2 | 3.2 |
| drying property | 3 seconds | 10 minutes | 20 minutes | 10 minutes | X*1 | 1 hour | 12 hours |
| transparenty | ◯ | ◯ | ◯ | ◯ | ◯ | ◯ | ◯ |
| water resistance | ◯ | ⊚ | ◯ | ⊚ | ◯ | ◯ | ⊚ |
| oil resistance | ◯ | ◯ | ⊚ | ⊚ | X | ◯ | ◯ |
| adhesiveness | ⊚ | ⊚ | ⊚ | ◯ | X | ◯ | ◯ |
| spreadability | ⊚ | ⊚ | ⊚ | ◯ | ◯ | X | X |
| stretch feeling | ⊚ | ⊚ | ⊚ | ◯ | ◯ | X | X |

*1: Though the composition was left in the atmosphere for three days after application, the composition was in a liquid state and did not form a film.

Preparation of cosmetic liquid for sunscreen

### Example 5

A cosmetic for sunscreen was prepared by mixing 89 parts by weight of a liquid composition according to the present invention obtained in the above described Example 3 and 11 parts by weight of ethanol.

### Comparative Example 7

A cosmetic liquid for sunscreen was prepared by mixing the following components at the following ratio.

TABLE 4

| Component | Comparative Example 7 |
|---|---|
| 1) octamethylcyclotetra siloxane | 41 |
| 2) butylmethoxydibenzoyl methane | 2 |
| 3) octylmethoxycinnamic acid | 7 |
| 4) dimethylpolysiloxane | 34 |
| 5) decamethylcyclopenta siloxane | 5 |
| 6) ethanol | 11 |

Preparation of cosmetic milky liquid for sunscreen

### Example 6

A milky liquid for sunscreen was prepared by heating a liquid composition obtained in Example 2 to 70° C., adding to the composition and mixing it with 8 parts by weight of particulate titanium oxide and 4 parts by weight of particulate zinc oxide, and slowly adding ion-exchange water and glycerine while stirring the mixture. Each component and the blend ratio are summarized in the following Table.

### Comparative Example 8

Instead of a liquid composition according to Example 2, a mixture liquid was used which was prepared by mixing 22.5 parts by weight of decamethylcyclopenta siloxane, 5 parts by weight of liquid paraffin, 1.5 parts by weight of dimethylpolysiloxane (50cps), 5 parts by weight of octylmethoxy cinnamate, paraben and a small amount of perfume, a mixture liquid. Then, the mixture liquid was heated to 70° C. to dissolve the components thereof. Subsequently, 10 parts by weight of particulate titanium oxide and 10 parts by weight of particulate zinc oxide were added to the heated mixture liquid, and the mixture was mixed. Furthermore, 30 parts by weight of ion-exchange water and 10 parts by weight of glycerine were slowly added thereto while stirring the mixture, to thus prepare a milky liquid for sunscreen. Each component and the blend ratio are summarized in the following Table.

### Comparative Example 9

Instead of a liquid composition according to Example 2, a mixture liquid was used which was prepared by mixing 14 parts by weight of decamethylcyclopenta siloxane, 5 parts by weight of liquid paraffin, 10 parts by weight of the polymer obtained after the precipitation and deposition in Comparative Example 5, 5 parts by weight of octylmethoxy cinnamate, paraben and a small amount of perfume. Then, the mixture liquid was heated to 70° C. to dissolve the components. Subsequently, 15 parts by weight of particulate titanium oxide was added to the heated mixture liquid, and the mixture was mixed. Furthermore, 30 parts by weight of ion-exchange water and 10 parts by weight of glycerine were slowly added thereto while stirring the mixture to thus prepare a milky liquid for sunscreen. Each component and the blend ratio are summarized in the following Table.

TABLE 5

| | | Comparative | Comparative |
|---|---|---|---|
| (component) | Example 6 | Example 8 | Example 9 |
| liquid composition in Example 2 | 43 | — | |
| polymer obtained in | — | — | 10 |
| Comparative Example 5 | | | |
| 1) decamethylpentasiloxane | — | 22.5 | 14 |
| 2) liquid paraffin | — | 5 | 5 |
| 3) dimethylpolysiloxane | — | 1.5 | |
| 4) octylemethoxy cinnamate | — | 5 | 5 |
| 5) paraben | — | appropriate | appropriate |
| | | amount | amount |
| 6) perfume | — | appropriate | appropriate |
| | | amount | amount |
| 7) particulate titanium oxide | 8 | 10 | 15 |
| 8) particulate zinc oxide | 4 | 10 | |
| 9) ion-exchange water | 30 | 30 | 30 |
| 10) glycerol | 10 | 10 | 10 |

Evaluation for performance of cosmetic liquid for sunscreen and cosmetic milky liquid for sunscreen

The following tests were conducted on the cosmetic liquid for sunscreen and the cosmetic milky liquid for sunscreen obtained in Examples 5 and 6 and Comparative Examples 7 and 9, in order to evaluate the performances of them.

(Evaluation Method)

1) Adhesiveness to skin

Several droplets of each cosmetic liquid or cosmetic milky liquid were applied onto the back of a hand with a finger, and dried. Then, the formed film was rubbed with a finger, and a degree of adhesiveness of the film was judged from the peeled state of the film.

When the film was not peeled off by rigorous rubbing, the adhesiveness was evaluated to be Excellent, ⊚; when a part of the film was peeled off by three or more times of rubbing, the adhesiveness was evaluated to be Good, ◯; and when the film was completely peeled off by one or two times of rubbing, the adhesiveness was evaluated to be Poor, ×.

2) Transparency

Several droplets of each cosmetic liquid or cosmetic milky liquid were applied onto the back of a hand, and was dried. The transparency was evaluated by whether or not there was white bluring on the skin.

When the white blur was not recognized at all, the transparency was evaluated to be Excellent, ⊚; when the white blur was slightly recognized, the transparency was evaluated to be Good, ◯; and when the white blur was clearly recognized, the transparency was evaluated to be Poor, ×.

3) Spread

Several droplets of each cosmetic liquid or cosmetic milky liquid were applied onto the back of a hand, and were spread with a finger until the liquid was dried. The spread was evaluated by whether or not there was a caught feeling as spread.

When there was no caught feeling, the spread was evaluated to be Excellent, ⊚; when there was some caught feeling but the composition could be spread, the spread was evaluated to be Good, ◯; and when there was caught feeling immediately after the composition started to be spread with a finger, and the composition was not spread, the spread was evaluated to be Poor, ×.

4) Stretch feeling and creaky feeling

Several droplets of each cosmetic liquid or cosmetic milky liquid was applied onto the back of a hand, and was naturally dried to form a coating film. Then, the stretch feeling was evaluated by a sense of a subject.

When there was no stretch feeling, the stretch feeling was evaluated to be Excellent, ⊚; when the subject felt uncomfortable due to some stretch feeling, the stretch feeling was evaluated to be Good, ◯; and when the subject felt strong stretch feeling, the stretch feeling was evaluated to be Poor, ×.

5) Fresh feeling

Several droplets of each cosmetic liquid or cosmetic milky liquid was applied onto the back of a hand, and was naturally dried to form a coating film. Then, the presence or absence of stickiness and the slippery feeling of the skin were evaluated by a sense of a subject.

When the subject felt the coating film no sticky and silky feeling, the fresh feeling was evaluated to be Excellent, ⊚; when the subject felt the coating film somewhat sticky, the fresh feeling was evaluated to be Good, ◯; and when the subject felt the coating film unpleasant due to stickiness, the fresh feeling was evaluated to be Poor, ×.

6) Water resistance

Several droplets of each cosmetic liquid or cosmetic milky liquid were applied onto the back of a hand. Two hours after, the parts of hands having a coating film were washed with water while being rubbed to each other for three minutes. Then, the water resistance was judged from the condition of the parts.

When the coating film was not washed off at all, the water resistance was evaluated to be Excellent, ⊚; when a part of the coating film was washed off, the water resistance was evaluated to be Good, ◯; and when the whole coating film was washed off, the water resistance was evaluated to be Poor, ×.

7) Oil resistance

Several droplets of each cosmetic liquid or cosmetic milky liquid were applied onto the back of a hand. Two hours after, n-dodecane was rubbed onto the parts of hands having a coating film with a finger for about 1 minute. Then, the oil resistance was judged from the condition of the parts.

When the coating film was not rubbed off at all, the oil resistance was evaluated to be Excellent, ⊚; when a part of the coating film was rubbed off, the oil resistance was evaluated to be Good, ◯; and when the whole coating film was rubbed off, the oil resistance was evaluated to be Poor, ×.

8) Ultraviolet-blocking effect

Several droplets of each cosmetic liquid or cosmetic milky liquid were applied onto the back of a hand on a bright ultraviolet-drenched day in May. Then, the subject played fishing on the shore all the day, and the ultraviolet-blocking effect was judged from the presence or absence of irritation due to sunburn.

When the irritation was not caused at all, the ultraviolet-blocking effect was evaluated to be Excellent, ⊚; when the irritation was not caused but the skin got dark, the ultraviolet-blocking effect was evaluated to be Good, ◯; and when the irritation was caused to make the skin reddish, the ultraviolet-blocking effect was evaluated to be Poor, ×.

TABLE 6
(text result)

| | Table 4 evaluation result | | | | |
|---|---|---|---|---|---|
| | | | Comparative | | |
| | Example 5 | 6 | Example 7 | 8 | 9 |
| adhesiveness to skin | ⊚ | ⊚ | ◯ | ◯ | ◯ |
| transparency | ◯ | ◯ | ⊚ | Δ | ◯ |
| spread | ⊚ | ⊚ | ⊚ | ◯ | X |
| stretch feeling | ⊚ | ⊚ | ⊚ | ◯ | X |
| fresh feeling | ⊚ | ⊚ | Δ | Δ | Δ |
| water resistance | ⊚ | ⊚ | Δ | ◯ | ◯ |
| oil resistance | ⊚ | ⊚ | X | Δ | ◯ |
| ultraviolet-blocking effect | ⊚ | ⊚ | Δ | ◯ | ⊚ |

Preparation of liquid bandage preparation

### Example 7

A liquid bandage preparation was prepared by adding 60 g of hexamethyldisiloxane to 40 g of a liquid composition obtained in Example 1, stirring and mixing them.

### Comparative Example 10

A liquid bandage preparation was prepared by adding 60 g of hexamethyldisiloxane to 40 g of a dissolution type silicone acrylic resin composition obtained in Comparative Example 6, stirring them and mixing them.

Evaluation for performance of liquid bandage preparation

A liquid bandage preparation obtained in Example 7 was sprayed onto a scratch on the back of a hand on a bright day in May. Pain immediately vanished, and after having been dried, the bandage preparation did not cause uncomfortable feeling such as stretch feeling in the vicinity of the scratch. Subsequently, the subject continued an operation outdoors all through the day, but irritation due to sunburn was not caused. The bandage preparation was sprayed twice a day. The scratch vanished on the third day without forming a scab.

In addition, in order to confirm a coating state of the liquid bandage preparation on the skin, we sprayed a liquid bandage preparation according to Example 7 on a pig skin and taken a photograph of the formed coating film at the magnification of 3,000 times with a scanning electron microscope manufactured by Hitachi, Ltd. We confirmed that a shape of the skin surface such as folds was transcribed onto the coating film as it was, and the liquid bandage preparation according to Example 7 formed a coating film with the surface shape precisely corresponding to the shape of the skin such as the folds. Such a shape of the coating film supports the fact that the formed film does not cause stretch feeling and has superior adhesiveness to the skin.

On the other hand, having sprayed a liquid bandage preparation according to Comparative Example 10 onto the back of a hand, the dried coating film caused stretch feeling and uncomfortable feeling. In addition, having rubbed the dried coating film with a finger six hours after the spraying, the coating film was peeled off in a film form.

Further, in order to confirm a coated state of the liquid bandage preparation on the skin, we sprayed a liquid bandage preparation according to Comparative Example 10 on a pig skin and taken a photograph of the formed coating film at the magnification of 3,000 times with a scanning electron microscope. We did not confirm that a shape of the skin surface such as folds was transcribed onto the coating film.

### Industrial Applicability

As described above, the present invention can provide a liquid composition of which the coated film on the skin is transparent and slippery, does not cause stickiness and uncomfortable feeling such as creaky feeling and stretch feeling, but results in fresh feeling of use, and keeps the capability of protecting the skin from ultraviolet rays for a desired period of time even under the presence of sweat or sebum, and which is suitable for cosmetics for sunscreen, a skin external preparation or a bandage preparation.

The present invention can provide a method for producing the liquid composition having such excellent characteristics.

1. A non-aqueous liquid composition comprising particles having ultraviolet-absorbing capability and silicone oil in which the particles are dispersed,
wherein the particles are composed of a copolymer derived from a silicone macromer (A) and a vinyl monomer (B) mainly containing a (meth)acrylate monomer and an organic ultraviolet absorber (C-1), and
wherein the silicone macromer (A) exists in the copolymer within a ratio of 1 to 20 wt % of all components of the particle and orients toward the outer surface of the particles.

2. A non-aqueous liquid composition comprising particles having ultraviolet-absorbing capability and silicone oil in which the particles are dispersed,
wherein the particles have a shell layer composed of a copolymer derived from a silicone macromer (A) and a vinyl monomer (B) mainly containing a (meta)acrylate monomer, and a core layer composed of a (co)polymer mainly derived from a (meta)acrylate monomer and an ultraviolet absorber (C-1), and
wherein the silicone macromer (A) exists in the copolymer within a ratio of 1 to 20 wt % of all components of the particles, and orients toward the outer surface of the particles.

3. A non-aqueous liquid composition comprising a particle having ultraviolet-absorbing capability and silicone oil in which the particles are dispersed,
wherein, the particles are composed of a copolymer deriving from a silicone macromer (A), a vinyl monomer (B) mainly containing a (meth)acrylate monomer and an ultraviolet-absorbing monomer (C-2), and
wherein the silicone macromer (A) exists in the copolymer within a ratio of 1 to 20 wt % of all components of the particles and orients toward the outer surface of the particle.

4. A non-aqueous liquid composition comprising a particle having ultraviolet-absorbing capability and silicone oil in which the particles are dispersed,
wherein the particles have a shell layer composed of a copolymer derived from a silicone macromer (A) and a vinyl monomer (B) mainly containing a (meth)acrylate monomer, and a core layer composed of a copolymer derived from a vinyl monomer (B) mainly containing a (meth)acrylate monomer and an ultraviolet-absorbing monomer (C-2); and
wherein the silicone macromer (A) exists in the copolymer within a ratio of 1 to 20 wt % of all components of the particles and orients toward the outer surface of the particle.

5. The non-aqueous liquid composition according to any one of claims 1 to 4,
wherein the silicone macromer (A) is a macromer represented by the general Formula 1:
[Formula 1] 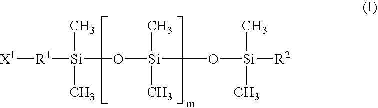
wherein R¹ represents a linear or branched alkylene group having 1 to 10 carbon atoms; R² represents a linear or branched alkylene group having 1 to 10 carbon atoms; m represents an integer of 3 to 300; and X represents a radical-polymerizable group.

6. The non-aqueous liquid composition according to any one of claims 3 to 4,
wherein the ultraviolet-absorbing monomer (C-2) is represented by the general Formula 2:
[Formula 2] 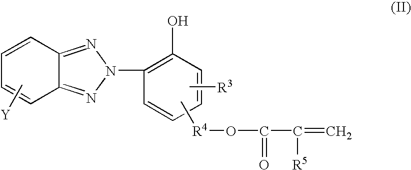
wherein R³ represents a hydrogen atom or an alkyl group having 1 to 10 carbon atoms; R⁴ represents a linear or branched alkylene group having 1 to 6 carbon atoms; R⁵ represents a hydrogen atom or a methyl group; and Y represents hydrogen atom, a halogen, an alkoxy group having 1 to 4 carbon atoms, a cyano group or a nitrile group.

7. The non-aqueous liquid composition according to any one of claims 1 to 4, wherein the surface of the particle has a contact angle with water of 90 to 120 degrees.

8. The non-aqueous liquid composition according to any one of claims 1 to 4, wherein the particles have an average particle size of 0.01 to 2 μm.

9. The non-aqueous liquid composition according to any one of claims 1 to 4, which comprises the silicone oil in an amount of 1 to 150 parts by weight to 100 parts by weight of the particles.

10. The non-aqueous liquid composition according to any one of claims 1 to 4, which has a solution viscosity of 1,000 mPa·s or less when having a solid content of 50 wt %.

11. The non-aqueous liquid composition according to any one of claims 1 to 4, wherein the organic ultraviolet absorber (C-1) or an ultraviolet-absorbing monomer (C-2) has a surface tension of 28 mN/m or higher when measured before polymerization.

12. A method for producing a non-aqueous liquid composition containing particles having ultraviolet-absorbing capability and silicone oil in which the particles are dispersed, which comprise subjecting the mixture of 1 to 20 wt % of a silicone macromer (a), 20 to 98 wt % of a vinyl monomer (b) mainly containing (meta)acrylate, and 1 to 60 wt % of an organic ultraviolet absorber (c-1) or an ultraviolet-absorbing monomer (c-2) to one or more radical polymerization reaction steps in the silicone oil.

13. A sunscreen cosmetic, a skin external preparation or a bandage preparation comprising a non-aqueous liquid composition according to any one of claims 1 to 4.
